(19) United States
(12) Patent Application Publication
(10) Pub. No.: US 2019/0046235 A1
(43) Pub. Date: Feb. 14, 2019

(54) BONE NEEDLE CLAMPS AND CANNULATED NEEDLE PINS FOR USE AS SKELETAL INFUSION NEEDLES AND METHODS THEREIN

(71) Applicants: Marc Waisman, Kiryat Bialik (IL); Gabriel Waisman, Tel Aviv (IL)

(72) Inventors: Marc Waisman, Kiryat Bialik (IL); Gabriel Waisman, Tel Aviv (IL)

(21) Appl. No.: 16/068,700

(22) PCT Filed: Jan. 24, 2017
(86) PCT No.: PCT/IL2017/050086

§ 371 Date: Jul. 9, 2018

Related U.S. Application Data
(60) Provisional application No. 62/292,878, filed on Feb. 9, 2016.

(51) Int. Cl. A61B 17/34; A61B 17/28

(52) U.S. Cl. CPC A61B 17/3472; A61B 17/282; A61B 2017/2808; A61M 2202/10; A61B 2017/2837

(57) ABSTRACT

The present invention discloses bone needle clamps and cannulated needle pins for use as skeletal infusion needles and methods therein. Bone needle clamps for facilitating intraosseous (IO) fluid transfer include: a clamp assembly having: at least one pair of opposing tips, facing each other, with each of the tips having a sharp end; and a bore in each tip, wherein the bore has a connection-facing opening and a bone-facing opening at opposing ends of the bore, and wherein the bone-facing opening is disposed in the tip at least one millimeter from the sharp end of each tip; and a needle assembly having: a cannula integrally attached to the connection-facing opening of the bore of the clamp assembly; and a connector operable to releasably attach a fluid-transfer assembly; wherein the needle assembly is operable to facilitate 10 infusion to the subject's bone and/or bone-marrow (BM) aspiration from the subject's bone.

### FIELD AND BACKGROUND OF THE INVENTION

The present invention relates to bone needle clamps and cannulated needle pins for use as skeletal infusion needles and methods therein. Such medical devices, and methods therein, allow access to the vascular system of a vertebrate.

Intraosseous (IO) infusion and bone marrow (BM) transfusion was first introduced in the 1940s, and was especially used in emergency cases during World War II, to avoid the lengthy times needed for finding a suitable blood vessel for insertion of a needle. In current times, the method is well known.

Rapid fluid administration is potentially life-saving in cardiopulmonary resuscitation (CPR), circulatory collapse, dehydration, diabetic ketoacidosis, burns, hypovolemic shock, and other trauma or medical emergencies needing to be treated with high venous fluid flow immediately or in the shortest time as possible. Choosing a central vein or inappropriate peripheral vein often causes complications or treatment delay which can easily be avoided by IO infusion. Several international guidelines state that IO or BM intravascular access should be obtained if there is inability to achieve a reliable venous access after three attempts or 90 seconds, whichever comes sooner.

The marrow cavity provides access to non-collapsible venous plexus as blood flows from the medullary venous sinusoids, and is then drained into the central venous circulation via nutrient and emissary veins. IO vascular access has proven to be an effective alternative to failed peripheral intravenous (IV) access in emergency cases in infants, children, and adults. Crystalloids, colloids, medications, or blood, delivered via IO vascular access, immediately diffuses into systemic circulation through the BM vein network. Such procedures are now preferred over an endrotracheal route for drugs during advanced life support in children and adults, including military and disaster medicine. BM aspiration is a reliable source of sampling for diagnostic purposes similar to blood testing.

Existing devices, whether manual or power-driven (in which a subjects bone size is evaluated to determine the length of the needle insertion), suffer from incorrect depth penetration mainly in children, difficult bone penetration in young adults, liquid extravasation, needle dislodgement (mainly from osteopenic, osteoporotic, or infant bone), and insufficient flowrate due to small needle gauge, and few suitable skeletal sites for bone infusion.

Elaborating on some of these shortcomings, penetration to the correct depth is a non-trivial task. Young patients have thin bone cortexes, while many elderly patients have fragile bones from conditions such as osteoporosis. Such patients occasionally suffer from micro-fractures around the site of the insertion cortex. Conversely, young adults often have dense bones that may resist penetration, even with power-driven devices. Moreover, the insertions are susceptible to extravasation due to difficulty with bone-cortex injuries related to needle fixation after penetration.

Furthermore, in conditions such as hypovolemia, the flowrate of replenishing fluid is often found lacking due to the insufficient needle gauge. Flowrates are related to the viscosity of the fluid, pressure gradient across the tubing, and the length and diameter of the tubing (Poiseue ̆s law). Typically, a 15-gauge needle is the largest needle that is used in adults, while an 18-gauge needle that is used in children. Such needles can provide a maximum flowrate of about 30-50 cc/min. for crystalloid fluids while applying infusion pressure. For a higher-viscosity fluid like blood, a maximum flowrate of only about 10-20 cc/min. can be obtained while applying infusion pressure.

Some patients are intimidated by the use of power-driven devices; however, manual insertion is frequently prone to spurious movements, and thereby excess damage to, and inadequate movement on the bone cortex. Finally, use of the commercially-available systems for penetrating the bone is restricted to a handful of suitable skeletal sites for the insertions (specifically limited to the proximal tibial metaphysis, distal tibial metaphysis, and proximal humeral metaphysis), some of which nevertheless are prone to complications.

In the patent literature, Waisman (U.S. Pat. No. 5,591,188) teaches a surgical instrument for inserting a trocar needle into the bone marrow of a patient for connection of a syringe, an infusion set or the like. The instrument includes a cylindrical housing containing a sliding bolt and the trocar needle configured to be forcefully shot through the bone material into the marrow by release of a compressed helical spring.

Flint (U.S. Pat. No. 7,135,031) teaches a single-shot trocar-needle insertion instrument configured for repeated use. The repeated-use trocar-needle insertion gun (RUTI) operates in association with a loading tool, which for reloading, is coupled with the RUTI via a loading opening.

Miller (US Patent Publication No. 2005/0171504) recites an apparatus and method for penetrating the bone marrow having a housing, a penetrator assembly, operable to penetrate the bone marrow, a connector operable to releasably attach the penetrator assembly to a drill shaft, the drill shaft operable to connect the penetrator assembly to a gear assembly, a gear assembly operable to engage and rotate the drill shaft, a motor operable to engage the reduction gear assembly and drive the penetrator into the bone marrow by rotation of the drill shaft, and a power supply and associated circuitry operable to power the motor.

Miller et al. (US Patent Publication No. 2008/0045857) recites various devices and methods to aspirate bone marrow from an associated bone using a powered drive and an aspiration needle or aspiration needle set. The aspiration devices may include a coupler assembly, a containment bag or sterile sleeve, an ejector, and/or an ejector funnel. A n aspiration needle set may include a cannula and trocar with respective tips having optimum configurations, dimensions and/or orientations relative to each other to optimize penetration of a bone and/or bone marrow with minimum trauma to a patient.

Miller et al. (US Patent Publication No. 2008/0045965) recites apparatus and methods to remove biopsy specimens from a bone and/or associated bone marrow using a powered drive and an IO needle set. The powered driver may rotate the IO needle set at an optimum speed to obtain a biopsy sample of bone and/or bone marrow.

Shoemaker (U.S. Pat. No. 5,725,532) teaches a non-opposed surgical reduction clamp which has a drill guide integrally formed with a guide leg of the reduction clamp.

Uwaydah (EP Application No. 0792621A1) recites a clamping device, having a guiding member with a guiding channel therethrough, wherein the guiding channel has an exit port and a receiving member operatively connected to the guiding member, wherein the receiving member has a receiving device in alignment with the exit port of the guiding channel so that a needle can be guided out of the exit port of the guiding channel; and into the receiving device of the receiving member.

Linvatec (U.S. Pat. No. 7,175,632) teaches a two-piece graft fixation arrangement having a graft block engageable with a graft and a transverse member engageable with the graft block to fix the graft block in a bone tunnel.

Kraus et al. (U.S. Pat. No. 6,340,361) teaches an external fixator system having a clamp adapted to couple a fixator pin to a connecting rod. The clamp includes a slot for transversely receiving the connecting rod. A bolt is inserted through a bore passing transversely to the slot to engage a pin connector holding a fixator pin. The pin connector has a rod-engaging surface that wedges the connecting rod into the slot thus increasing the clamps rigidity by preventing rotation of the clamp around the rod and rotation of the pin connector in the clamp body.

Raskin (US Patent Publication No. 2004/0133211) recites a radially-ported bone graft needle, particularly useful in minimally-invasive procedures. The bone graft needle delivers bone graft material to a bone defect area by extruding the bone graft material both axially and radially simultaneously.

It would be desirable to have bone needle clamps and cannulated needle pins for use as skeletal infusion needles and methods therein. Such medical devices and methods therein would, inter alia, overcome the various limitations mentioned above.

### SUMMARY

It is the purpose of the present invention to provide bone needle clamps and cannulated needle pins for use as skeletal infusion needles and methods therein. Such medical devices and methods provide for improved intraosseous (IO) access.

It is noted that the term ‘exemplary_is used herein to refer to examples of embodiments and/or implementations, and is not meant to necessarily convey a more-desirable use-case. Similarly, the terms ‘alternative_{—} and ‘alternatively_{—} are used herein to refer to an example out of an assortment of contemplated embodiments and/or implementations, and is not meant to necessarily convey a more-desirable use-case. Therefore, it is understood from the above that ‘exemplary_{—} and ‘alternative_{—} may be applied herein to multiple embodiments and/or implementations. Various combinations of such alternative and/or exemplary embodiments are also contemplated herein.

Embodiments of the present invention provide optimized self-fixation devices in the form of bone needle clamps. Such devices provide better liquid flow through the needle tips (e.g., up to ˜200 cc/min. under infusion pressure pump or pressure bag with 300 mm Hg through the adult proximal tibial metaphysis) and easier, smoother bone penetration of the needle tips, preventing extravasation. Such bone needle clamps can be used in a greater variety of bone insertion sites, and provide better fixation.

Embodiments of the present invention provide cannulated needle pins that provide better liquid flow through the needles (e.g., ˜200-300 cc/min., with higher flowrates being suitable for veterinary use). Furthermore, such cannulated needle pins provide means for better fixation when used in conjunction with additional cannulated needle pins. Such multi-pin configurations also enable ‘dual delivery_{—} of fluids for even greater flowrates or simultaneous delivery of two fluids. Moreover, since operational use of such cannulated needle pins is implemented in a ‘through-bone_{—} configuration in which the pins both enter and exit (from the opposite side) the subjects bone, there is no need to measure or predetermine insertion/penetration depth.

Therefore, according to the present invention, there is provided for the first time a bone needle clamp for facilitating intraosseous (IO) fluid transfer, the bone needle clamp including: (a) a clamp assembly having: (i) at least one pair of opposing tips, the opposing tips facing each other, with each of the tips having a sharp end; and (ii) a bore in each tip, wherein the bore has a connection-facing opening and a bone-facing opening at opposing ends of the bore, and wherein the bone-facing opening is disposed in the tip at least one millimeter from the sharp end of each tip; wherein the clamp assembly is operable to penetrate a subjects bone when the tips are employed to exert force against the subjects bone during closure of the tips of the clamp assembly and thereby provide self-fixation; and (b) a needle assembly having: (i) a cannula integrally attached to the connection-facing opening of the bore of the clamp assembly; and (ii) a connector operable to releasably attach a fluid-transfer assembly; wherein the needle assembly is operable to facilitate IO infusion to the subjects bone and/or bone-marrow (BM) aspiration from the subjects bone.

Alternatively, the connector includes a Luer lock.

Alternatively, the fluid-transfer assembly is at least one component selected from the group consisting of: a syringe, an infusion set, a medication set, an infusion pressure pump, and a pressure bag.

Alternatively, the bone needle clamp further includes: (c) a closure assembly, integrally attached to the clamp assembly, for releasably controlling a closure amount of the closure of the tips of the clamp assembly.

Most alternatively, the closure assembly is at least one assembly selected from the group consisting of: a ratchet securing mechanism and a bar/wingnut securing mechanism.

Alternatively, the clamp assembly and the needle assembly are operable to provide a fluid flowrate of up to about 200 cc/minute to the subject ̆s bone for IO infusion or to obtain a BM aspiration sample for diagnostic purposes.

Alternatively, the clamp assembly and the needle assembly are operable on the subject ̆s bone via at least one bone-insertion site selected from the group consisting of: a proximal tibial metaphysis, a medial malleolus, a distal tibial metaphysis, a lateral malleolus, a calcaneus, a first metatarsal, a greater trochanter, a proximal humeral metaphysis, an olecranon, a distal radius posterior metaphysis, a first metacarpus distal metaphysis, a first proximal phalanx proximal metaphysis, a second metacarpus distal metaphysis, a fifth metacarpus distal metaphysis, and an anterior superior iliac spine.

According to the present invention, there is provided for the first ti me a method for facilitating intraosseous (IO) fluid transfer, the method including the steps of: (a) penetrating a subjects bone using a bone needle clamp, wherein the bone needle clamp has: (i) a clamp assembly, the clamp assembly including: (A) at least one pair of opposing tips, the opposing tips facing each other, with each of the tips having a sharp end; and (B) a bore in each tip, wherein the bore has a connection-facing opening and a bone-facing opening at opposing ends of the bore, and wherein the bone-facing opening is disposed in the tip at least one millimeter from the sharp end of each tip; wherein the clamp assembly is operable to penetrate a subject ̆s bone when the tips are employed to exert force against the subject ̆s bone during closure of the tips of the clamp assembly and thereby provide self-fixation; and (ii) a needle assembly, the needle assembly including: (A) a cannula integrally attached to the connection-facing opening of the bore of the clamp assembly; and (B) a connector operable to releasably attach a fluid-transfer assembly; wherein the needle assembly is operable to facilitate IO fluid transfer to and/or from the subjects bone; and (b) transferring at least one fluid using the bone needle clamp for IO infusion to the subjects bone and/or for bone-marrow (BM) aspiration from the subjects bone.

Alternatively, the fluid-transfer assembly is at least one component selected from the group consisting of: a syringe, an infusion set, a medication set, an infusion pressure pump, and a pressure bag.

Alternatively, the step of transferring is operable to provide a fluid flowrate of up to about 200 cc/minute to the subject ̆s bone for IO infusion or to obtain a BM aspiration sample for diagnostic purposes.

Alternatively, the subjects bone includes at least one bone-insertion site selected from the group consisting of: a proximal tibial metaphysis, a medial malleolus, a distal tibial metaphysis, a lateral malleolus, a calcaneus, a first metatarsal, a greater trochanter, a proximal humeral metaphysis, an olecranon, a distal radius posterior metaphysis, a first metacarpus distal metaphysis, a first proximal phalanx proximal metaphysis, a second metacarpus distal metaphysis, a fifth metacarpus distal metaphysis, and an anterior superior iliac spine.

According to the present invention, there is provided for the first time a cannulated needle-pin assembly for facilitating intraosseous (JO) fluid transfer, the cannulated needle-pin assembly including: (a) a cannula including: (i) a connection-facing end operable to releasably attach a first fluid-transfer assembly; (ii) a bone-facing end having a sharp tip operable to engage a subjects bone upon penetration; (iii) at least one proximal fluid entrance/exit hole disposed along the cannula in communication with a hollow interior of the cannula; and (iv) at least one distal fluid entrance/exit hole disposed in close proximity to the sharp tip, at least one distal fluid entrance/exit hole in communication with the hollow interior operable to releasably attach a second fluid-transfer assembly.

Alternatively, the cannula further includes: (v) at least one marking band on an exterior of the cannula operable to identify an extent of insertion indicative of a bone-penetration depth upon penetration into the subjects bone.

Alternatively, the cannula further includes: (v) a connector operable to releasably attach the second fluid-transfer assembly.

Most alternatively, the connector includes a Luer lock.

Alternatively, the first fluid-transfer assembly and the second fluid-transfer assembly are independently at least one component selected from the group consisting of: a tubing, a syringe, an infusion set, a medication set, an infusion pressure pump, and a pressure bag.

Alternatively, the cannula is operable to provide a fluid flowrate in at least one subject application selected from the group consisting of: up to about 200 cc/minute in a human-subject application and up to about 300 cc/minute in an animal-subject application.

Alternatively, the cannula is operable on the subjects bone via at least one bone-insertion site selected from the group consisting of: a femur distal supracondylar distal metaphysis, a proximal tibial metaphysis, a distal tibial metaphysis, and a distal radius posterior metaphysis.

Alternatively, the cannula is operable to provide dual fluid delivery in a through-bone configuration, with both the connection-facing end and the bone-facing end external to the subject ̆s bone, in which the first fluid-transfer assembly and the second fluid-transfer assembly are employed to administer different fluids via the subjects bone.

Alternatively, the cannula is operable to provide simultaneous fluid delivery in a through-bone configuration, with both the connection-facing end and the bone-facing end external to the subjects bone, in which the first fluid-transfer assembly and the second fluid-transfer assembly are employed to administer similar fluids via the subject ̆s bone.

Alternatively, the cannulated needle-pin assembly further includes: (b) a stylette operable to be disposed along an interior of the cannula in order to stiffen the cannula during insertion into the subject ̆s bone.

Alternatively, the cannulated needle-pin assembly further includes: (b) a pin-fixation clasp operable to be engaged on external portions of at least two cannulae in a multi-pin configuration in order to provide enhanced pin fixation, wherein the external portions are external to the subject ̆s bone.

According to the present invention, there is provided for the first time a method for facilitating intraosseous (IO) fluid transfer, the method including the steps of: (a) penetrating a subjects bone using a cannulated needle-pin assembly, wherein the cannulated needle-pin assembly has: (i) a cannula including: (A) a connection-facing end operable to releasably attach a first fluid-transfer assembly; (B) a bone-facing end having a sharp tip operable to engage the subjects bone upon penetration; (C) at least one proximal fluid entrance/exit hole disposed along the cannula in communication with a hollow interior of the cannula; and (D) at least one distal fluid entrance/exit hole disposed in close proximity to the sharp tip, at least one distal fluid entrance/exit hole in communication with the hollow interior operable to releasably attach a second fluid-transfer assembly; and (b) transferring at least one fluid using the cannulated needle-pin assembly to the subjects bone for IO infusion and/or from the subjects bone for bone-marrow (BM) aspiration.

Alternatively, the cannula further includes: (E) at least one marking band on an exterior of the cannula operable to identify an extent of insertion indicative of a bone-penetration depth upon penetration into the subjects bone.

Alternatively, the first fluid-transfer assembly and the second fluid-transfer assembly are independently at least one component selected from the group consisting of: a tubing, a syringe, an infusion set, a medication set, an infusion pressure pump, and a pressure bag.

Alternatively, the step of transferring is operable to provide a fluid flowrate in at least one subject application selected from the group consisting of: up to about 200 cc/minute in a human-subject application and up to about 300 cc/minute in an animal-subject application. Alternatively, the subjects bone includes at least one bone-insertion site selected from the group consisting of: a femur distal supracondylar distal metaphysis, a proximal tibial metaphysis, a distal tibial metaphysis, and a distal radius posterior metaphysis.

Alternatively, the step of transferring is operable to provide dual fluid delivery with the step of penetrating being performed in a through-bone configuration, with both the connection-facing end and the bone-facing end external to the subjects bone, in which the first fluid-transfer assembly and the second fluid-transfer assembly are employed to administer different fluids via the subjects bone.

Alternatively, the step of transferring is operable to provide simultaneous fluid delivery with the step of penetrating being performed in a through-bone configuration, with both the connection-facing end and the bone-facing end external to the subjects bone, in which the first fluid-transfer assembly and the second fluid-transfer assembly are employed to administer similar fluids via the subjects bone.

Alternatively, the cannulated needle-pin assembly further has: (ii) a stylette operable to be disposed along an interior of the cannula in order to stiffen the cannula during the step of penetrating.

Alternatively, the cannulated needle-pin assembly further has: (ii) a pin-fixation clasp operable to be engaged on external portions of at least two cannulae with the step of penetrating being performed with at least two cannulae in a multi-pin configuration in order to provide enhanced pin fixation, wherein the external portions are external to the subject ̆s bone. These and further embodiments will be apparent from the detailed description and examples that follow.

### BRIEF DESCRIPTION OF THE DRAWINGS

The present invention is herein described, by way of example only, with reference to the accompanying drawings, wherein:

FIG. 1a depicts a bone needle clamp having a pair of clamp tips, each with a tip-coupled cannula aligned with a bore extending through and exiting each tip and a ratchet securing mechanism as part of the handle, according to embodiments of the present invention;

FIG. 1b depicts a close-up view of the bone needle clamp of FIG. 1a magnifying one of the tips with its cannula and bore, according to embodiments of the present invention;

FIG. 1c depicts an alternative perspective view of the bone needle clamp of FIG. 1a illustrating the curvature of the clamp ̆s tips, according to embodiments of the present invention;

FIG. 1d depicts a modified version of the bone needle clamp of FIG. 1a having a bar/wingnut securing mechanism as part of the handle, according to alternative embodiments of the present invention;

FIG. 1e depicts the bone needle clamp of FIG. 1a in operational use, according to embodiments of the present invention;

FIG. 2 depicts an alternative perspective view of the bone needle clamp of FIG. 1a in which the clamp ̆s tips are hollow having a sharp end with each cannula sheathed in the tips connected to Luer locks, according to embodiments of the present invention;

FIG. 3a depicts a perspective view of a cannulated pin having a sharp end and entrance/exit holes that are in communication with a hollow pin, according to embodiments of the present invention;

FIG. 3b depicts a longitudinal section-view of the cannulated pin of FIG. 3a as configured during insertion use, according to embodiments of the present invention;

FIG. 3c depicts a longitudinal section-view of a tube that is configured to allow the passage of fluids via entrance/exit holes at the sharp end of the cannulated pin of FIG. 3a, according to embodiments of the present invention;

FIG. 3d depicts more than one of the cannulated pins of FIG. 3a being implemented for use at a selected skeletal-bone area of a subject in order to enhance fixation when engaged with a pin clasp, according to embodiments of the present invention.

### DESCRIPTION OF THE ILLUSTRATIVE EMBODIMENTS

The present invention relates to bone needle clamps and cannulated needle pins for use as skeletal infusion needles and methods therein. The principles and operation for providing such medical devices and methods, according to the present invention, may be better understood with reference to the accompanying description and the drawings.

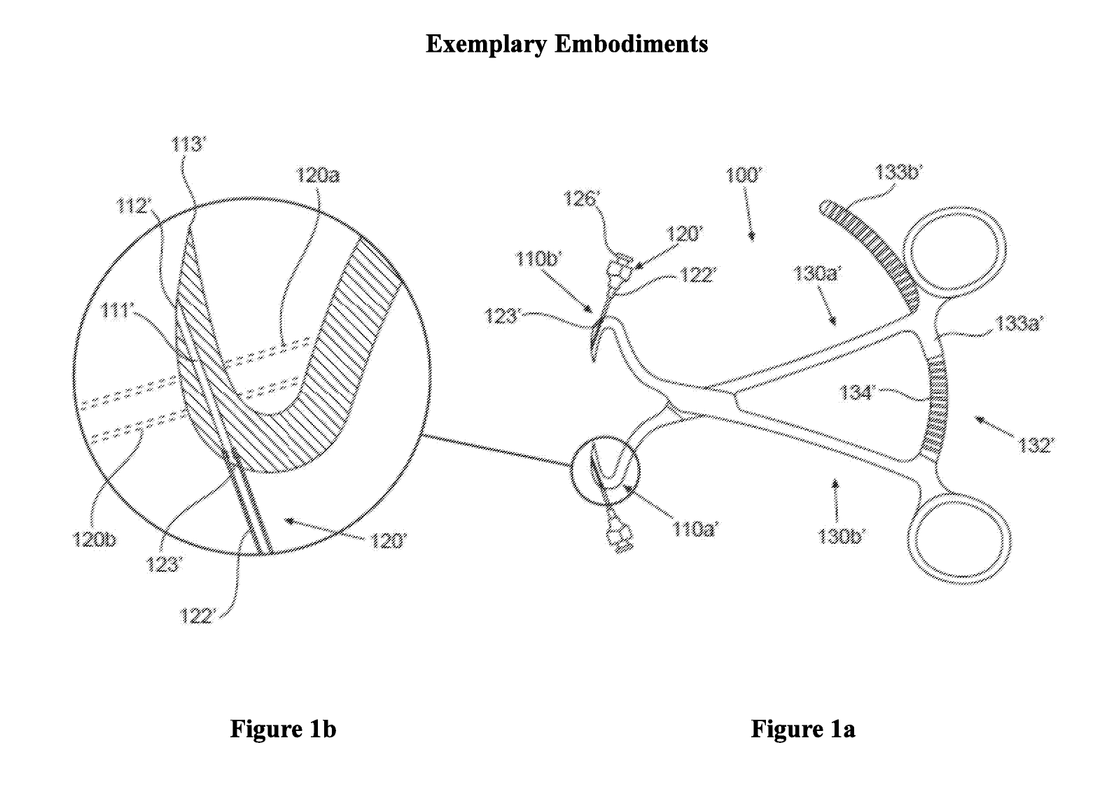

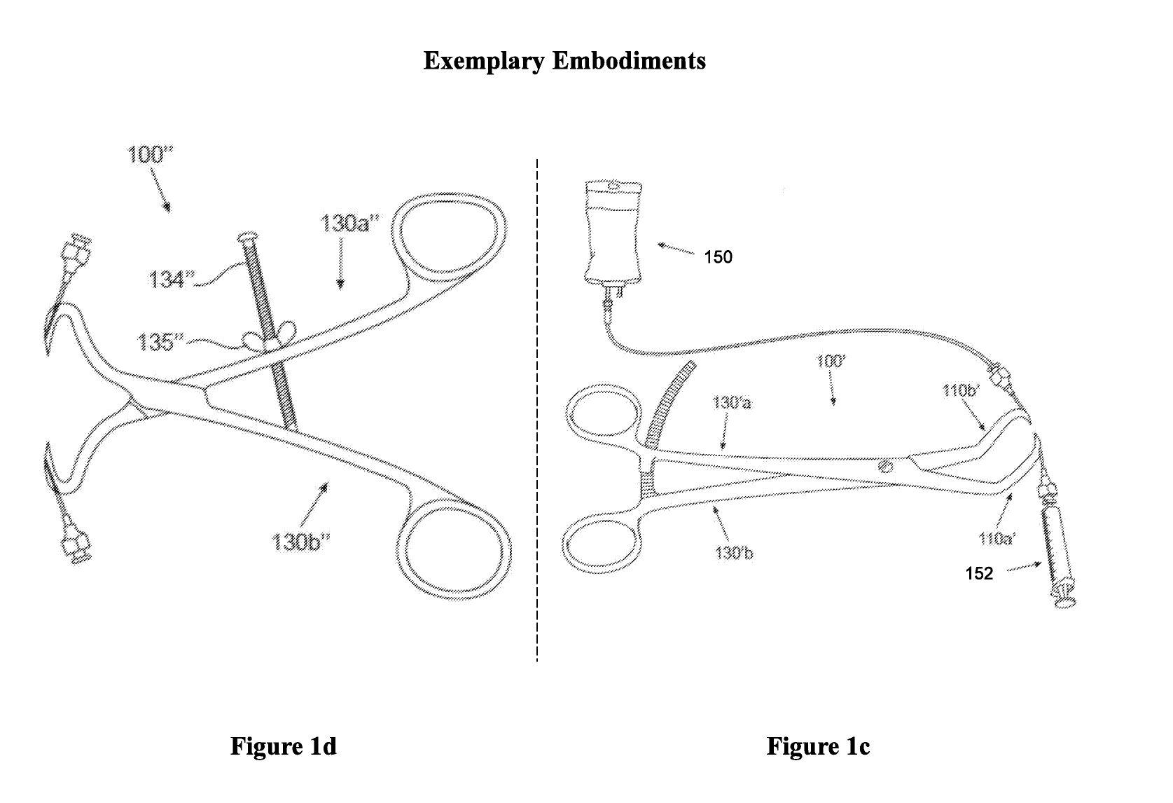

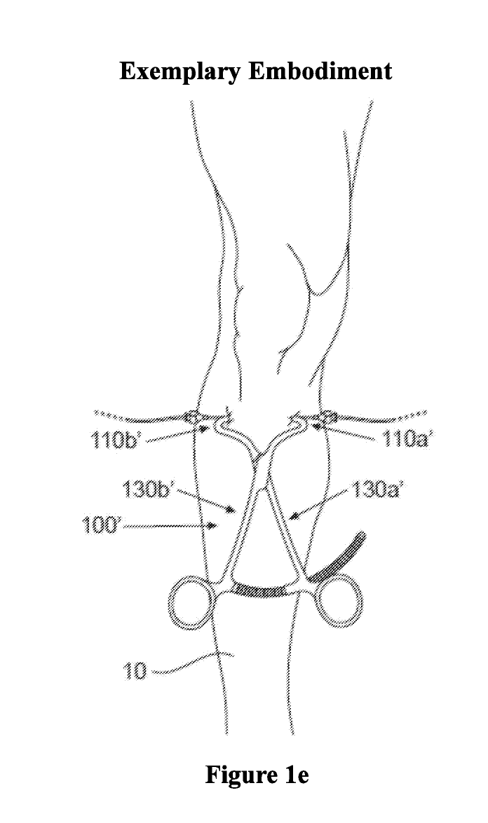

Referring to the drawings, FIG. 1a depicts a bone needle clamp having a pair of clamp tips, each with a tip-coupled cannula aligned with a bore extending through and exiting each tip and a ratchet securing mechanism as part of the handle, according to embodiments of the present invention. A bone needle clamp 100′ having a pair of essentially opposing tips including a first tip 110′a and a second tip 110b′. A needle 120′ is coupled to each tip, enabling the delivery of fluids to an area engaged by bone needle clamp 100′. Needle 120′ is shown having a cannula 122′ (i.e., a pipe made of metal or other rigid material that doesn't deform upon bone penetration) typically with a pointed end 123′. A Luer lock 126′ at or near the free end of cannula 122′ enables connection of syringes and/or infusion equipment to cannula 122′ for fluid transfer (i.e., fluid delivery to via the bone for IO infusion or fluid extraction for BM aspiration, such as for obtaining a sample for diagnostic purposes).

FIG. 1b depicts a close-up view of the bone needle clamp of FIG. 1a magnifying one of the tips with its cannula and bore, according to embodiments of the present invention. End 123′ typically conforms essentially in orientation to the slope of tip 110′ at the area of contact therebetween. Cannula 122′ has an internal diameter that is substantially larger than a bore 111′ in each of tips 110a′ and 110b′ such that cannula 122′ can be welded or otherwise firmly affixed to the tip exterior surrounding bore 111′ (i.e., the tip exterior is a ‘connection-facing_{—} opening of the bore), with cannula 122’ and bore 111′ preferably being aligned. Bore 111′ preferably has openings on the convex side of each of tips 110a′ and 110b′ as shown. In alternative embodiments, bore 111′ extends close to the sharpest point on each of tips 110a′ and 110b′, with bore 111′ having an opening 112′ (i.e., a ‘bone-facing_{—} opening) substantially ˜1 mm or more away from a sharpest apex 113’ on each of tips 110a′ and 110b′. It is preferable to maintain such distances between the bore opening and the apex of the tip in order to avoid potential plugging of the bore due to bone blockage of the tip upon insertion.

Both tips 110a′ and 110b′ are inserted into a subjects bone to the extent shown in the dashedlines of FIG. 1b. A dashed line 120a represent the bone cortex and a dashed line 120b represents the skin line. Clamp 100′ may be sterilized between uses (e.g., in an autoclave) or intended for disposable use. Luer lock 126′ may be sealed to preserve sterility with a suitable cap (not shown).

Bone needle clamp 100′ of FIG. 1a generally has the shape and dimension of common Stagbeetle Bone Reduction Forceps, in particular with regard to the curvature of each of tips 110a′ and 110b′ and the length of handles 130a′ and 130b′ relative to the size of each of tips 110a and 110b′. Bone needle clamp 100′ is configured to allow tips 110a′ and 110b′ to open relative to each other to a wide angle and distance, typically over 90é, in order to allow bone needle clamp 100′ to engage bones having a wide range of sizes. Bone needle clamp 100′ of FIG. 1a further includes a ratchet 132′ which allows the setting of the opening between handles 130a′ and 130b′. Ratchet 132′ has a notched bar 133a′ and 133b′ on each of handles 130a′ and 130b′, with notches 134′ facing each other and interlocking when handles 130a′ and 130b′ are closed.

FIG. 1c depicts an alternative perspective view of the bone needle clamp of FIG. 1a illustrating the curvature of the clamp ̆s tips, according to embodiments of the present invention. In exemplary embodiments of bone needle clamp 100′, tips 110a′ and 110b′ are both curved relative to the plane defined by handles 130a′ and 130b′, with apices 113 of tips 110a′ and 110b′ facing each other. An infusion bag 150 and a syringe 152 are shown in FIG. 1c connected to bone needle clamp 100′.

FIG. 1d depicts a modified version of the bone needle clamp of FIG. 1a having a bar/wingnut securing-mechanism as part of the handle, according to alternative embodiments of the present invention. A bone needle clamp 100″ is shown in a top view in FIG. 1d. Handles 130a″ and 130b″ have a threaded bar 134″ and a wingnut 135″ instead of ratchet 132′ of bone needle clamp 100′ in FIG. 1a. First handle 130a″ has a hole therethrough for accommodating bar 134″, and second handle 130b″ has a recess (not shown) in which bar 134″ is affixed to second handle 130b″ to allow first handle 130a″ and second handle 130b″ to freely move apart when wingnut 135″ is fully opened on bar 134″.

In alternative embodiments (not shown), more than one pair of needle tips can be employed in a configuration of one pair of tips being disposed behind the other to allow for increased fluid throughput. In other embodiments (not shown), a bone needle clamp can be deployed with a conventional bone clamp in a configuration of the conventional clamp being disposed behind (i.e., on top of) the needle clamp to allow for enhanced fixation of the needle clamp.

FIG. 1e depicts the bone needle clamp of FIG. 1a in operational use, according to embodiments of the present invention. When tips 110a and 110b″ of bone needle clamp 100′ penetrate a bone during operational use on a selected skeletal-bone area 10 of a subject, handles 130a′ and 130b′ stabilize the position of bone needle clamp 100′. Typically, first tip 110a′ penetrates the bone more than second, identical, opposing tip 110b′, or vice versa. Since tips 110a′ and 110b′ are tapered, as first tip 110a′ further penetrates the bone, resistance to further penetration increases until the resistance to first tip 110a′ becomes greater than the resistance to second tip 110b′. First tip 100a′ thereby serves as a base to provide a counterforce, and second tip 110b′ can then further penetrate into the bone.

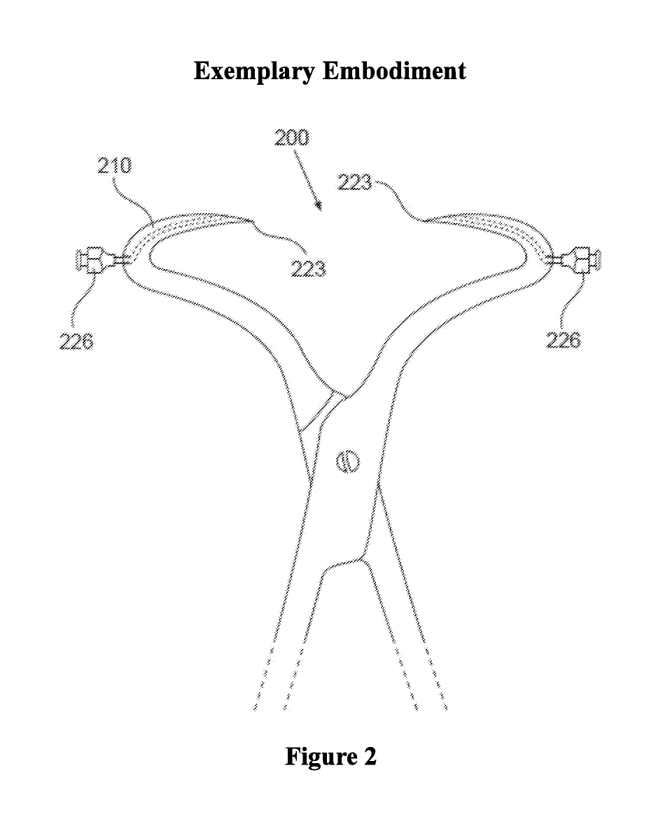

FIG. 2 depicts an alternative perspective view of the bone needle clamp of FIG. 1a in which the clamp ̆s tips are hollow having a sharp end with each cannula sheathed in the tips connected to Luer locks, according to embodiments of the present invention. A bone needle clamp 200 is shown in which tips 210 are each hollow and have a sharp apex 223. Each tip 210 is connected to a lateral short cannula 223 and a Luer lock 226.

Recommended insertion sites for the bone needle clamp described above include: (1) for the lower limb⁻ proximal tibial metaphysis, medial malleolus, distal tibial metaphysis, lateral malleolus, calcaneus, first metatarsal, and greater trochanter; (2) for the upper limb⁻ proximal humeral metaphysis, olecranon, distal radius posterior metaphysis, first metacarpus distal metaphysis, first proximal phalanx proximal metaphysis, second metacarpus distal metaphysis, and fifth metacarpus distal metaphysis; and (3) for other locations⁻ anterior superior iliac spine.

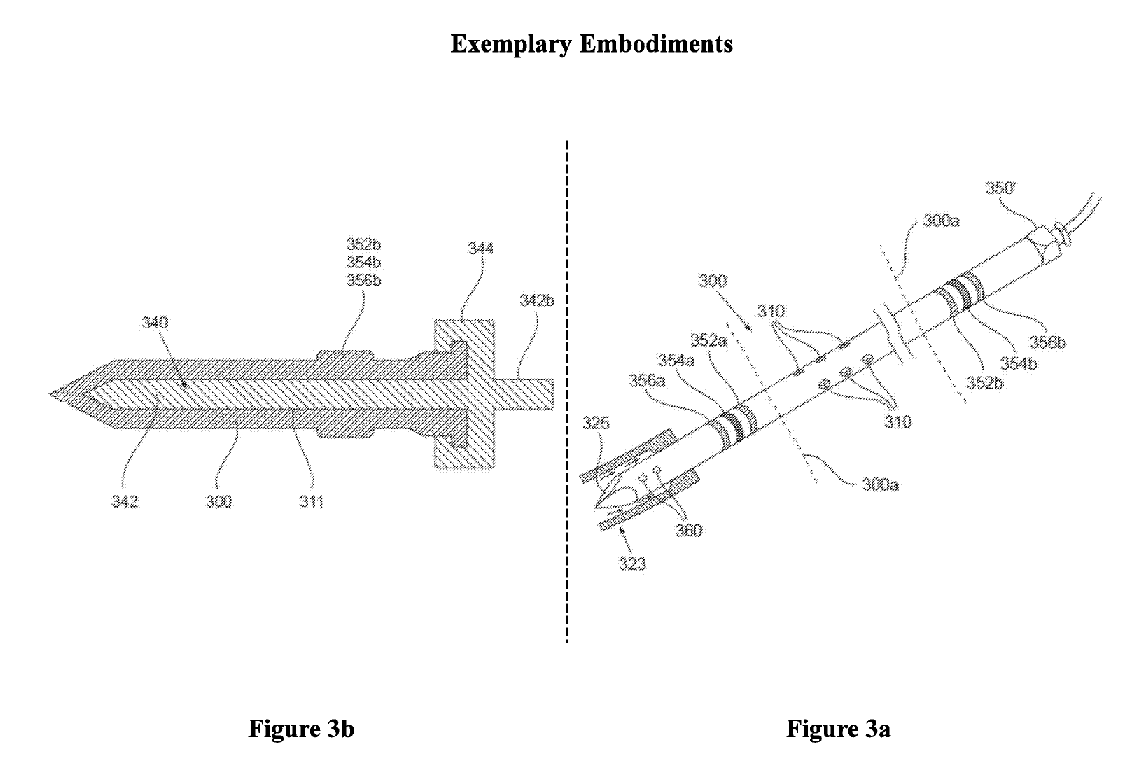

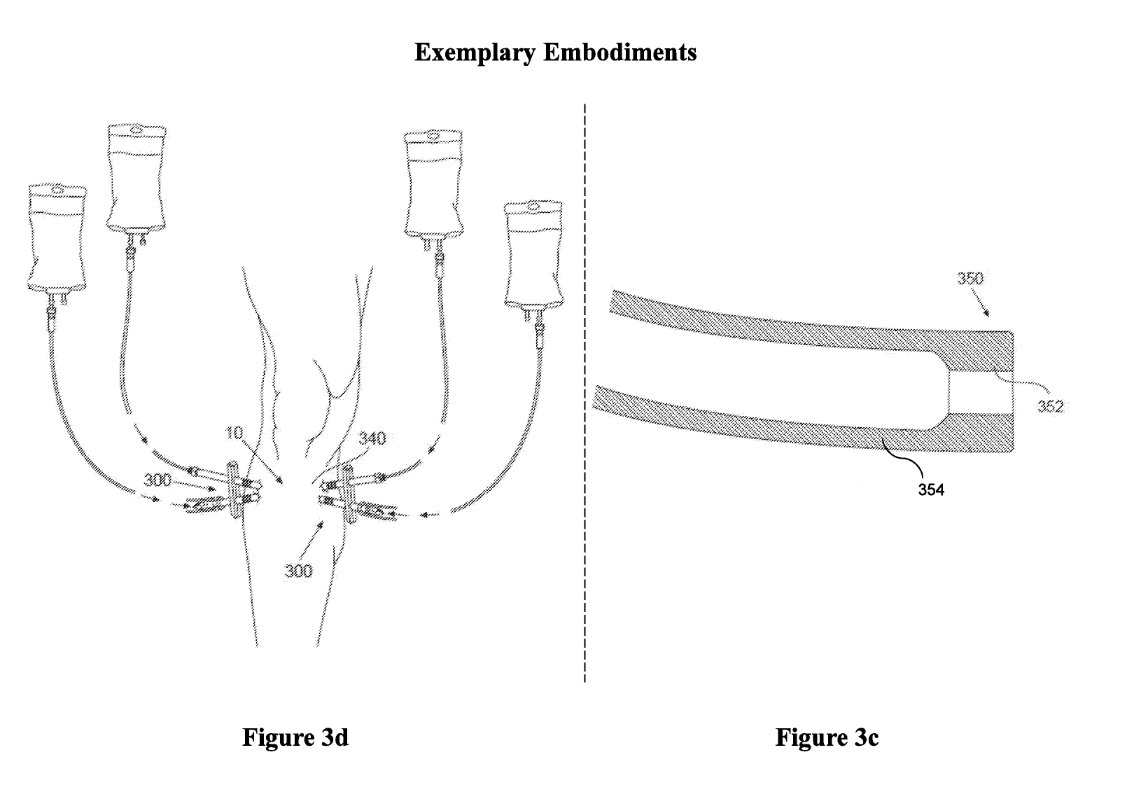

FIG. 3a depicts a perspective view of a cannulated pin having a sharp end and entrance/exit holes that are in communication with a hollow pin, according to embodiments of the present invention. An improved cannulated needle pin 300 having a hollow interior is shown with a sharp end 323 (i.e., a ‘bone-facing_{—} end) that preferably has at least two facets 325. Alternatively, cannulated needle pin 300 may have three or more facets 325. Facets 325 help to create firm engagement of cannulated needle pin 300 with a bone upon penetration during bone drilling or hammering. Cannulated needle pin 300 further includes entrance/exit holes 310 that are in communication with the hollow interior (not shown) of cannulated needle pin 300. Entrance/exit holes 310 are positioned along cannulated needle pin 300 to allow for maxi mal fluid flow into the B M through sinusoidal veins. Dashed lines 300a represent the bone cortex.

FIG. 3b depicts a longitudinal section-view of the cannulated pin of FIG. 3a as configured during insertion use, according to embodiments of the present invention. A stylette 340 is employed to augment the strength of cannulated needle pin 300. Stylette 340 includes a rod portion 342 that fits inside cannulated needle pin 300, preferably with a diameter only slightly smaller than the inner diameter of cannulated needle pin 300. Stylette 340 is adapted to easily slide out of cannulated needle pin 300 after insertion of the pin is completed. Stylette 340 further includes a cap 344 that covers an entrance 312 to a hollow interior 311. Force-transferring means 342b (e.g., a rod) can be coupled to cap 344 to allow force to be transferred to rod 342. Cap 344 overlaps cannulated needle pin 300 with a Luer lock closing to allow sterility of hollow interior 311 to be maintained, and to enable engagement of cap 344 with rod portion 342 to be stabilized.

Returning to FIG. 3a, progress of the insertion of cannulated needle pin 300 into bone can be evaluated by bands 352a, 352b, 354a, 354b, 356a and 356b, for example, which are disposed on cannulated needle pin 300 on either side of entrance/exit holes 310. The bands may be paired (e.g., bands 352a and 352b; bands 354a and 354b; bands 356a and 356b) by color, pattern, and/or a metal small wing, for example. The bands are preferably equidistant from the longitudinal center of cannulated needle pin 300. Thus, the pin may be visually centered in a bone. Band 352a, close to the center of cannulated needle pin 300, may serve as an absolute limit to insertion in order to prevent the leakage of fluids outside the bone and over-insertion. Cannulated needle pin 300 further includes entrance/exit holes 360 which are close to facets 325. On the other side (i.e., a ‘connection-facing_{—} end) of cannulated needle pin 300, a Luer lock 350′ is provided through which liquids can be introduced using an infusion-set connector or a syringe.

FIG. 3c depicts a longitudinal section-view of a tube that is configured to allow the passage of fluids via entrance/exit holes at the sharp end of the cannulated pin of FIG. 3a, according to embodiments of the present invention. Part of a flexible tube 350 is shown, and serves to supply fluids to cannulated needle pin 300. At least one tube 350 is typically connected to cannulated needle pin 300 after the pin is fully inserted (according to the users determination of appropriate insertion depth). Tube 350 includes a thick end 352 that can be snugly fitted over cannulated needle pin 300 and a thinner part 354 (i.e., the part with thinner walls in FIG. 3c). Tube 350 is pulled over cannulated needle pin 300 until thick end 352 is positioned between entrance/exit holes 360 and band 356. In embodiments lacking bands, hick end 352 is positioned proximally after entrance/exit holes 360 such that liquid can pass through the thinner part 354 and into cannulated needle pin 300 without leakage.

FIG. 3d depicts more than one of the cannulated pins of FIG. 3a being implemented for use at a selected area of a subject in order to enhance fixation when engaged with a pin clasp, according to embodiments of the present invention. More than one cannulated needle pin 300 can be used at selected skeletal-bone area 10 of a subject Multiple cannulated needle pins 300 allow increased throughput of fluids. In addition, multiple cannulated needle pins 300 can be engaged together with a clasp 340 that restricts movement of the pins to avoid dislodgement and provide maxi mal pin fixation. Moreover, in such a configuration, multiple sources of liquids can be introduced through cannulated needle pins 300 in order to increase liquid flow.

Cannulated needle pins 300 may be provided in a kit. The kit may include a plurality of pins, perhaps of various sizes, as well as optional accessories such as one or more of the flexible tubes, and protective covers. Such a protective cover (not shown) may be placed on sharp end 323 of cannulated needle pin 300 after insertion in situations in which sharp end 323 juts out of the limb, as shown in FIG. 3d. The protective cover may then be placed on sharp end 323, and subsequently flexible tube 350 can be placed over sharp end 323 of cannulated needle pin 300.

Beyond fixation of cannulated needle pins 300 (which form a crisscross configuration in the limb to prevent unwanted movement of the pins), the two pins in the arrangement of FIG. 3d provide four different access points for fluid supply, thus allowing rapid and massive fluid supply which was previously unattainable. As shown in FIG. 3d, flexible tube 350 is shown to be placed on the tip of cannulated needle pin 300 in order to allow liquids to pass through entrance/exit holes 360 as shown by the illustrative arrows.

Such multi-pin configurations also enable ‘dual delivery_{—} of fluids for even greater flowrates or simultaneous delivery of two fluids. Moreover, since operational use of such cannulated needle pins is implemented in a ‘through-bone_{—} configuration in which the pins both enter and exit (from the opposite side) the subjects bone, there is no need to measure or predetermine insertion/penetration depth.

Recommended insertion sites for the cannulated pin include: (1) for the lower limb⁻ femur distal supracondylar distal metaphysis, proximal tibial metaphysis, and distal tibial metaphysis; and (2) for the upper limb⁻ distal radius posterior metaphysis.

While the present invention has been described with respect to a limited number of embodiments, it will be appreciated that many variations, modifications, equivalent structural elements, combinations, sub-combinations, and other applications of the present invention may be made.

1. A bone needle clamp for facilitating intraosseous (TO) fluid transfer, the bone needle clamp comprising:
a) a clamp assembly having:
i) at least one pair of opposing tips, said opposing tips facing each other, with each of said tips having a sharp end; and
ii) a bore in each said tip, wherein said bore has a connection-facing opening and a bone-facing opening at opposing ends of said bore, and wherein said bone-facing opening is disposed in said tip at least one millimeter from said sharp end of each said tip;
wherein said clamp assembly is operable to penetrate a subject's bone when said tips are employed to exert force against said subject's bone during closure of said tips of said clamp assembly and thereby provide self-fixation; and
b) a needle assembly having:
i) a cannula integrally attached to said connection-facing opening of said bore of said clamp assembly; and
ii) a connector operable to releasably attach a fluid-transfer assembly;
wherein said needle assembly is operable to facilitate IO infusion to said subject's bone and/or bone-marrow (BM) aspiration from said subject's bone.

2. The bone needle clamp of claim 1, wherein said connector includes a Luer lock.

3. The bone needle clamp of claim 1, wherein said fluid-transfer assembly is at least one component selected from the group consisting of: a syringe, an infusion set, a medication set, an infusion pressure pump, and a pressure bag.

4. The bone needle clamp of claim 1, the bone needle clamp further comprising:
c) a closure assembly, integrally attached to said clamp assembly, for releasably controlling a closure amount of said closure of said tips of said clamp assembly.

5. The bone needle clamp of claim 4, wherein said closure assembly is at least one assembly selected from the group consisting of: a ratchet securing mechanism and a bar/wingnut securing mechanism.

6. The bone needle clamp of claim 1, wherein said clamp assembly and said needle assembly are operable to provide a fluid flowrate of up to about 200 cc/minute to said subject's bone for IO infusion or to obtain a BM aspiration sample for diagnostic purposes.

7. The bone needle clamp of claim 1, wherein said clamp assembly and said needle assembly are operable on said subject's bone via at least one bone-insertion site selected from the group consisting of: a proximal tibial metaphysis, a medial malleolus, a distal tibial metaphysis, a lateral malleolus, a calcaneus, a first metatarsal, a greater trochanter, a proximal humeral metaphysis, an olecranon, a distal radius posterior metaphysis, a first metacarpus distal metaphysis, a first proximal phalanx proximal metaphysis, a second metacarpus distal metaphysis, a fifth metacarpus distal metaphysis, and an anterior superior iliac spine.

8. A method for facilitating intraosseous (TO) fluid transfer, the method comprising the steps of:
a) penetrating a subject's bone using a bone needle clamp, wherein said bone needle clamp has:
i) a clamp assembly, said clamp assembly including:
A) at least one pair of opposing tips, said opposing tips facing each other, with each of said tips having a sharp end; and
B) a bore in each said tip, wherein said bore has a connection-facing opening and a bone-facing opening at opposing ends of said bore, and wherein said bone-facing opening is disposed in said tip at least one millimeter from said sharp end of each said tip;
wherein said clamp assembly is operable to penetrate a subject's bone when said tips are employed to exert force against said subject's bone during closure of said tips of said clamp assembly and thereby provide self-fixation; and
ii) a needle assembly, said needle assembly including:
A) a cannula integrally attached to said connection-facing opening of said bore of said clamp assembly; and
B) a connector operable to releasably attach a fluid-transfer assembly;
wherein said needle assembly is operable to facilitate TO fluid transfer to and/or from said subject's bone; and
b) transferring at least one fluid using said bone needle clamp for TO infusion to said subject's bone and/or for bone-marrow (BM) aspiration from said subject's bone.

9. The method of claim 8, wherein said fluid-transfer assembly is at least one component selected from the group consisting of: a syringe, an infusion set, a medication set, an infusion pressure pump, and a pressure bag.

10. The method of claim 8, wherein said step of transferring is operable to provide a fluid flowrate of up to about 200 cc/minute to said subject's bone for TO infusion or to obtain a BM aspiration sample for diagnostic purposes.

11. The method of claim 8, wherein said subject's bone includes at least one bone-insertion site selected from the group consisting of: a proximal tibial metaphysis, a medial malleolus, a distal tibial metaphysis, a lateral malleolus, a calcaneus, a first metatarsal, a greater trochanter, a proximal humeral metaphysis, an olecranon, a distal radius posterior metaphysis, a first metacarpus distal metaphysis, a first proximal phalanx proximal metaphysis, a second metacarpus distal metaphysis, a fifth metacarpus distal metaphysis, and an anterior superior iliac spine.

12-31. (canceled)

32. The bone needle clamp of claim 1, wherein said needle assembly further has:
iii) a stylette operable to be reversibly disposed along an interior of said cannula in order to stiffen said cannula during said closure of said tips of said clamp assembly against said subject's bone.

33. The method of claim 8, wherein said needle assembly further includes:
C) a stylette operable to be reversibly disposed along an interior of said cannula in order to stiffen said cannula during said closure of said tips of said clamp assembly against said subject's bone.
